(19) United States
(12) Patent Application Publication
(10) Pub. No.: US 2024/0374341 A1
(43) Pub. Date: Nov. 14, 2024

(54) BLOOD PRESENCE DETECTION

(71) Applicant: Verb Surgical Inc., Santa Clara, CA (US)

(72) Inventors: Arjun Ravi Shankar, Santa Clara, CA (US); Rami Abhukhalil, Santa Clara, CA (US); Genevieve Foley, Santa Clara, CA (US); Varun Goel, Santa Clara, CA (US); Jocelyn Barker, Santa Clara, CA (US)

(21) Appl. No.: 18/196,961

(22) Filed: May 12, 2023

(51) Int. Cl. A61B 90/00; A61B 34/00

(52) U.S. Cl. CPC A61B 90/361; A61B 34/25; A61B 2090/364; G16H 30/40

(57) ABSTRACT

A surgery assessment system includes a camera, a processor or controller, and a display. The processor or controller is configured to receive at least one image collected at a surgery site by the camera, analyze the at least one image with a machine-learned model, and calculate a blood presence score for the surgery site based on the analysis of the at least one image. The display is configured to output the blood presence score in association with the surgery site.

### FIELD

The present application relates to the detection of blood at a surgical site.

### BACKGROUND

Minimally invasive surgery (MIS), such as laparoscopic surgery, involves techniques intended to reduce tissue damage during a surgical procedure. A surgeon may insert a laparoscope including a video camera and one or more instruments through a small opening cut in the human body. Without the video camera to monitor the surgical site, a larger opening would be required for the surgical procedure.

Bleeding that occurs during the surgery, referred to as intraoperative bleeding, can be problematic during minimally invasive surgery due to limited vision and mobility. The bleeding is not easily detected by the surgeon when the surgical site is not immediately visible. Intraoperative adverse events, of which inoperative bleeding is a part, may contribute to complications including death for 98,000 to 400,000 people each year. Intraoperative bleeding may impact post operative outcomes and lead to additional procedures or surgeries.

Challenges remain in accurately detecting intraoperative bleeding during minimally invasive surgery procedures.

### SUMMARY

By way of introduction, the preferred embodiments described below include methods, systems, instructions, and computer readable media for storing instructions for bleeding assessment at a surgery site including receiving at least one image collected at a surgery site, analyzing the at least one image with a machine-learned model, calculating a blood presence score for the surgery site based on the analysis of the at least one image, and outputting the blood presence score in association with the surgery site.

At least one embodiment also includes identifying a time period for the blood presence score, wherein the at least one image includes a plurality of images collected during the time period, and the blood presence score is based on an average of output values of the machine-learned model for the plurality of images.

At least one embodiment also includes identifying an instrument for the surgery site, associating the at least one image with the instrument, and assigning the blood presence score with the instrument.

At least one embodiment also includes comparing the blood presence score to an instrument threshold, and reporting instrument performance in response to the comparison.

At least one embodiment also includes identifying a medical professional for the surgery site, associating the at least one image with the medical professional, and assigning the blood presence score with the medical professional.

At least one embodiment also includes comparing the blood presence score to a user threshold, and reporting medical professional performance in response to the comparison.

At least one embodiment also includes accessing instructional information in response to the blood presence score, and providing the instructional information to the medical professional.

At least one embodiment also includes aggregating blood presence data including the blood presence score for a facility including the surgery site, and providing an assessment for the facility including a plurality of surgery sites.

At least one embodiment also includes identifying a bleeding location in at least one of the plurality of images, and modifying the at least one of the plurality of images to highlight the bleeding location.

At least one embodiment also includes determining one or more timestamps for the plurality of images, and assigning the one or more timestamps to the blood presence score. The timestamps may correspond to a part of the surgery or step in the procedure performed at the surgical site.

At least one embodiment also includes receiving instrument data associated with one or more instrument timestamps, and correlating the one or more instrument timestamps with the one or more blood presence timestamps.

At least one embodiment also includes collecting ground truth data for a plurality of training images, and training the machine-learned model based on the ground truth data.

At least one embodiment also includes identifying a surgery type for the surgery site, associating the at least one image with the surgery type, and assigning the blood presence score with the surgery type.

At least one embodiment includes a camera configured to collect a plurality of images collected at a surgery site, a processor configured to analyze the plurality of images with a machine-learned model and calculate a blood presence score for the surgery site based on the analysis of the plurality of images, and a display configured to output the blood presence score in association with the surgery site.

In at least one embodiment, the display provides a video including the plurality of images in association with the blood presence score. In at least one embodiment, output values of the machine-learned model for the plurality of images are averaged to calculate the blood presence score.

At least one embodiment also includes an instrument interface configured to receive instrument data for an instrument associated with the surgery site. In at least one embodiment, the display is configured to provide a plurality of blood presence scores for a plurality of surgeries.

At least one embodiment includes a surgery assessment system including a memory configured to store a plurality of images collected at a surgery site, and a processor configured to analyze the plurality of images with a machine-learned model and calculate a blood presence score for the surgery site based on the analysis of the plurality of images.

### BRIEF DESCRIPTION OF THE DRAWINGS

The embodiments of the invention are illustrated by way of example and not by way of limitation in the figures of the accompanying drawings in which like references indicate similar elements. It should be noted that references to “an” or “one” embodiment of the invention in this disclosure are not necessarily to the same embodiment, and they mean at least one. Also, in the interest of conciseness and reducing the total number of figures, a given figure may be used to illustrate the features of more than one embodiment of the invention, and not all elements in the figure may be required for a given embodiment.

FIGS. 1A-1C illustrates example surgical instruments;

FIG. 2A illustrates an example surgical site with significant bleeding;

FIG. 2B illustrates an example surgical site without significant bleeding;

FIG. 3A illustrates an example of a system for blood presence detection;

FIG. 3B illustrates another example of a system for blood presence detection;

FIG. 3C illustrates another example of a system for blood presence detection;

FIG. 4A illustrates example blood presence values for a surgical site without significant bleeding;

FIG. 4B illustrates example blood presence values for a surgical site with significant bleeding;

FIG. 5 illustrates an example blood presence score and warning;

FIG. 6 is a chart of various surgery types and statistical data for blood presence;

FIG. 7 illustrates an embodiment of the blood presence detection system; and

FIG. 8 illustrates an example flow chart for the blood presence detection system of FIG. 7.

### DETAILED DESCRIPTION

As described above, intraoperative bleeding is not easily detected by a surgeon or other medical professional when the surgical site is not immediately visible. Even when the site is immediately visible, the surgeon or medical professional cannot reliably assess or evaluate the amount of bleeding over time (e.g., aggregate bloodiness throughout the entire surgery). Bloodiness throughout the surgery is difficult to quantify post-surgery.

A model to predict the presence of blood in surgery enables surgeons to compare the bloodiness of their surgeries to other similar procedures. Such a model would also enable them to associate different surgical steps or surgical instruments with bloodiness. The following embodiments include apparatus and techniques to evaluate bleeding at a surgical site from a video including one or more images. The bleeding may be quantified into a blood presence score. The presence of blood, or to what degree, may be calculated using a machine learning model. The presence of blood, or to what degree, may be the basis of an annotation and/or label for regions of interest in the video of the surgical site. In some examples, a display subsequent to the surgery uses the blood presence score to advise surgeons or other medical professionals when, where, and possibly, why bleeding occurred. In some examples, a display subsequent to the surgery provides examples of surgical techniques to reduce bleeding during the particular type of surgery. In other examples, a system for a facility may utilize the blood presence score to evaluate surgical teams or team members. In other examples, a manufacturer or other entity may evaluate surgical instruments or devices according to the blood presence score. The minimally invasive surgical platform may provide feedback to the surgeon based on the blood presence score.

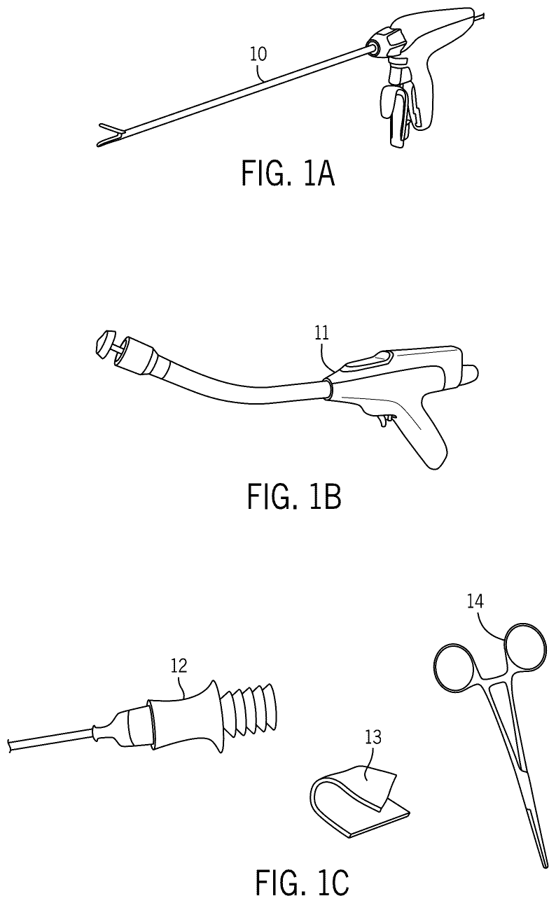

FIGS. 1A-1C illustrate hemostatic products configured to reduce or stop bleeding and/or perform at least one surgery function. FIG. 1A illustrates an energy device 10. The energy device 10 may include an electrosurgical generator to generate electrical energy or radio frequency signals that seal vessels or tissue in contact with the end of the instrument. The energy device 10 may include a jaw to grip and hold the tissue or vessel as well as apply energy to seal the tissue or vessel. The energy device 10 may include a battery pack or another power source.

FIG. 1B illustrates a stapler 11. Staples are loaded into the device and delivered to the tissue. A staple housing may be placed inside tissue opposite to an anvil that is placed in a lumen for stapling. The stapler 11 may include a battery pack or another power source for firing the stapler in cooperation with the anvil.

FIG. 1C illustrates passive hemostatic products including an adsorbable adjunctive hemostat applicator 12. a hemostat fabric 13, and hemostat forceps 14. The passive hemostatic products may include a powder that stops bleeding. The applicator 12 may dispense the powder at the surgical site. The fabric 13 may include powder particles that are placed in the surgical site. The hemostat forceps 14 may provide a clamping force on a vessel or other tissue to stop bleeding.

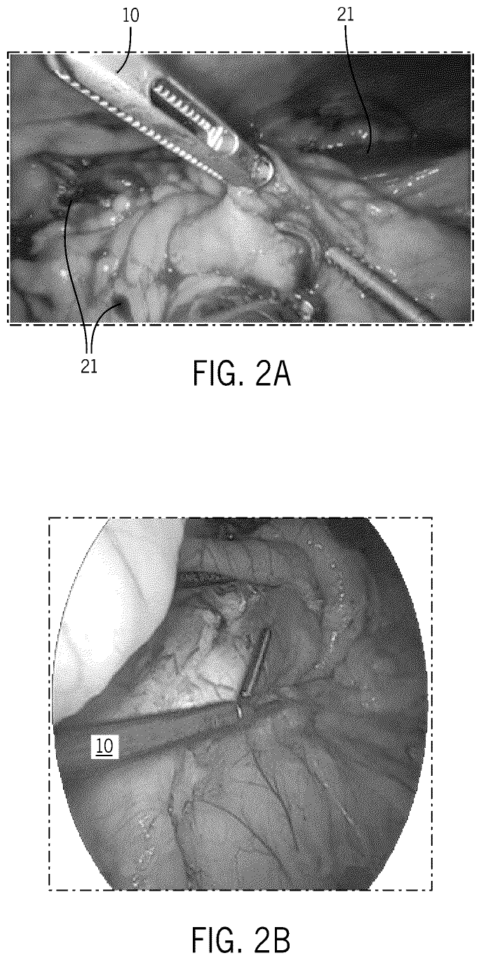

FIGS. 2A and 2B illustrate example images collected at a surgical site. The images may be collected by a video camera placed inside the human body. FIG. 2A illustrates an example surgical site with significant bleeding at several locations 21. FIG. 2B illustrates an example surgical site without significant bleeding. The images may be frames of a video. The images may include human tissue, organs, skin, or other parts of the human body. The images may include surgical instruments.

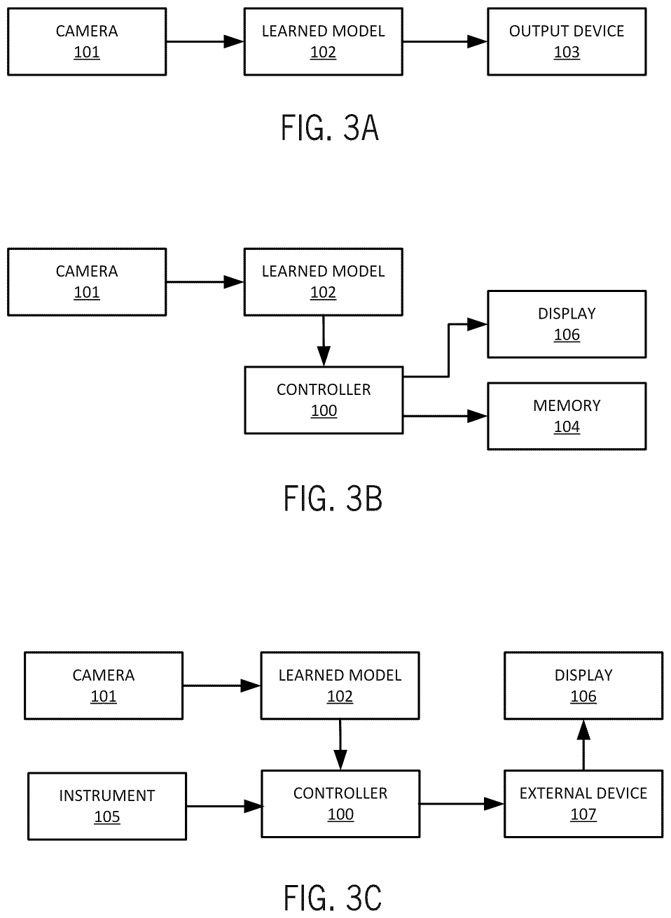

FIG. 3A illustrates an example of a system for blood presence detection. The system includes at least a camera 101, a learned model 102, hereinafter “model”, and an output device 103. Additional, different, or fewer components may be included.

The camera 101 may have various forms and implementations. The camera 101 may include at least one lens placed proximate to a window. The camera 101 may be coupled or otherwise associated with a light. Example lights may include cold light sources such as halogen or xenon. Light collected at the lens of the camera may be provided through a fiber or optical tube, which may extend through a cable or rod, to a video sensor that converts the light to electrical signals. Data may be transmitted from camera 101 through an electrical connection. Instructions may be received at the camera 101 through the electrical connection. Instructions may include a direction for the camera 101 achieved through actuation of a motor or through a pivot point in the cable or rod.

The camera 101 may be a laparoscope including a fiber optic cable system that is flexible and may be passed through an opening (incision or orifice) in the human body. The camera 101 may be passed through a cannula. The camera 101 may be coupled to a rod, such as a telescoping rod system.

The system may also be applied to other types of surgeries besides non-invasive surgeries. The surgical site could be an open site. The camera 101 may be mounted above patient such as on a stand. The camera 101 may have a direct view of the surgical site from the mounted location.

The model (e.g., learned model 102) receives at least one image collected at a surgery site by the camera 101. The model analyzes the at least one image according to parameters from training on past surgery images with known bloodiness. The model may be a feature prediction model includes a neural network or other machine learning/parallel processing system to automatically detect features in data items. The model may be a neural network having any number of layers. One example model for computer vision may be a residual neural network (ResNet) such as ResNet 18 with 18 layers. The model may include one or more convolutional layers. The model may include one or more feedforward paths or skips.

The neural network may be a computer vision model that is trained on images or video frames of surgical video as ground truth. The model may determine blood presence data for the surgery site based on the analysis of the at least one image. That is, the images may be labeled according to blood presence (e.g., perceived blood presence). The blood presence, which may be referred to as a blood presence state or blood presence characteristics, may be classified or labeled as blood present, blood absent, not sure, or flagged. The term not sure may refer to the absence of a classification (e.g., indeterminate, or inconclusive). The term flagged may mean that another issue was identified such as the image includes poor picture quantity, the image includes something other than a surgical site, the file is corrupt or another error. In another example, the blood presence may be classified or labeled as blood present or blood absent.

In another example, the output of the model or the blood presence value may be a numerical value. The numerical value may correspond to a percentage or a probability such as a confidence level. For example, for a given image, the model may output a blood presence value of 0.75 or 75, which indicates a 75% probability or likelihood that the video under analysis by the model has blood present above a predetermined threshold.

The learned model 102 was previously trained using machine learning. Machine learning is an offline training phase where the goal is to identify an optimal set of values of learnable parameters of the learned model 102 that can be applied to many different inputs (i.e., previously unseen inputs for a given video frame or image).

The neural network may be a fully connected neural network or a convolutional neural network. As another example, a regression model, such as linear, logistic, ridge, Lasso, polynomial, or Bayesian linear regression model is used. Any architecture or layer structure for machine learning may be used, such as U-Net, encoder-decoder, or image-to-image network. The architecture defines the structure, learnable parameters, and relationships between parameters. Different machine-learned models may be used for different outputs, such as different models or a multi-task model to generate different internal regions.

The model may be machine trained. Deep or other machine training is performed. Many (e.g., hundreds or thousands) samples of inputs to the model and the ground truth outputs are collected as training data. For example, video data from testing from surgeries performed on patients is collected as training data. The inputs are measured, and the resulting output is measured through inspection of the images or video from surgery. Simulation may be used to generate the training data. Experts may curate the training data, such as assigning ground truth for collected samples of inputs.

The ground truth data may be determined by many human annotators. The annotators may receive images or partial images, and the multiple annotators may receive the same images. The results of the annotators may be filtered or otherwise modified statistically to remove outliers. Weights may be used based on the performance of the annotators.

The machine training optimizes the values of the learnable parameters. Backpropagation, RMSprop, ADAM, or another optimization is used in learning the values of the learnable parameters of the model. Where the training is supervised, the differences (e.g., L1, L2, or mean square error) between the estimated output (e.g., centroid location and radius) and the ground truth output are minimized.

The model may provide a neural network output to output device 103. Many possibilities are possible for the output device 103. The output device 103 may report, transmit, or store a blood presence value or cumulative blood presence score with an identifier for the surgery. The identifier may be manually entered or automatically determined according to instance, user, or device. The output device 103 may log and record the blood presence values according to any of the examples and identifiers herein. Thus, the output of the learned model 102 may be a series of blood presence values or a running count of the blood presence values. Thus, during analysis of the images, the model outputs blood presence values over time and the output device 103 aggregates a total count of the blood present values. The total count of the blood presence values may be the blood presence score for the video. Likewise, a percentage of the total blood presence values count over the total number of samples may be the blood presence score.

In addition or alternatively, the output device 103 may communicate (or retain) a more detailed record of the analysis of the model. For example, the output device 103 may output (or store) the blood presence value with a timestamp for when the associated one or more images were collected. The output device 103 may store the blood presence value with the one or more images. The timestamp may correspond to a step in the procedure or surgery. The step in the procedure or surgery may be identified from the video. The step in the procedure or surgery may be identified from the presence, location, or actuation of a surgical instrument.

The output device 103 may include an alert. The alert may be a graphical object or alphanumerical character that is overlaid on the one or more images under analysis by the model. Thus, when the video is subsequently viewed, the alert is visible. The alert may trigger a light or audible alarm during the collection of the video (e.g., during the surgery). The alert may highlight a portion of the video (e.g., the source of the bleeding).

In another example, the output device 103 may determine whether the video under analysis includes high bloodiness or low bloodiness. The output device 103 may compare the count of the blood presence values to a threshold. When the count of the blood presence values exceeds the threshold, the output device 103 outputs data indicative of a bloody surgery. When the count of the blood presence values is less than the threshold, the output device 103 outputs data indicative of a substantially blood free surgery.

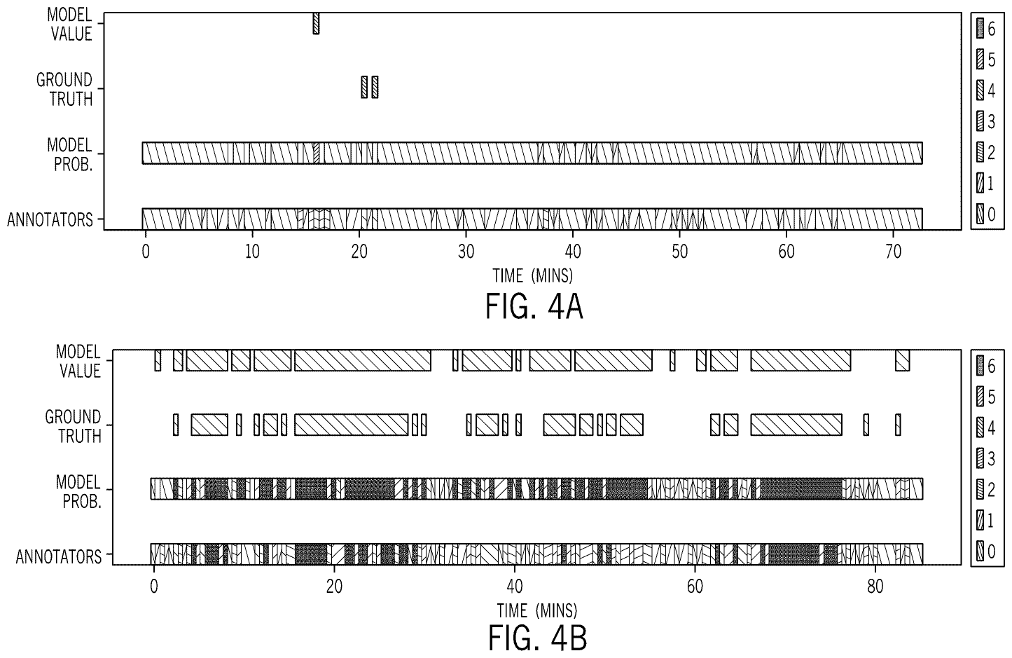

The output device 103 may generate and/or display an image indicative of the blood presence values. FIG. 4A illustrates example blood presence values for a surgical site without significant bleeding. FIG. 4B illustrates example blood presence values for a surgical site with significant bleeding. The Model Value plots in FIGS. 4A and 48 indicate blood presence values showing the presence of blood as a binary value (either present or not present). The Model Probability plots the probability value output from the model as represented by shading.

FIGS. 4A and 4B also show a comparison of the output of the model to the ground truth or human assessment (e.g., human annotators) of the same images. The Ground Truth plots compare to the binary values of the model to the human assessment of the same images. As shown in the mostly blood free surgery in FIG. 4A, most of the images are assessed the same by the model and the annotators. As shown in the mostly bloody surgery in FIG. 4B, most of the images are assessed the same by the model and the annotators.

The Annotators plots illustrate the number of human annotators (e.g., 0, 1, 2, 3, 4, 5, 6 shown in grayscale) that assess the individual frames as having blood as compared to the probability value output from the model.

FIG. 3B illustrates another example of a system for blood presence detection. The system includes at least a camera 101, a learned model 102, a controller 100, a display 106, and a memory 104, which may be integrated with or separate from the controller 100. Additional, different, or fewer components may be included.

The controller 100 performs calculations on the blood presence values or classifications. The blood presence values may be a probability number that the model 102 determines estimates the likelihood a given image includes the threshold amount of blood. The controller 100 performs a statistical analysis on the blood presence values. The blood presence values or statistical analysis for the blood presence score may be stored in memory 104. The blood presence values or statistical analysis for the blood presence score may be displayed at display 106, which may be integrated with controller 100 or a separate device (e.g., monitor, mobile device, etc.).

The controller 100 may average the blood presence values over multiple frames or images in order to calculate a blood presence score for the video. More specifically, the controller 100 is configured to analyze a set of blood presence values output from the model for a set of images, respectively. The set of images may be sequential. The set of images may be defined by a predetermined time period. The set of frames or images may be defined as the entire video. The set of frames or images may be defined as a certain interval in the video such as when an incision is made or a particular instrument is used. The set of frames or images may be defined according to a surgical step. The incision, instrument, or surgical step may be identified from analysis of the video. In one example, a second learned model is used to analyze the image and identify the incision, instrument, or surgical step.

The controller 100 may perform other techniques on the output of the model or blood presence values than averaging. For example, the controller 100 may first filter the blood presence values below a certain value (e.g., 0.05) so that certain frames are omitted. The controller 100 may identify a median value in the set of frames as an alternative blood presence score. The controller 100 may determine a number of frames over a set value (e.g., 0.80) as an alternative blood presence score. The controller 100 may calculate a variance or standard device of the blood presence values as an alternative blood presence score.

The computer vision model is applied to frames of new surgical videos. The probabilities outputted by the computer vision model are then averaged across a given video, surgical step, or other time period of interest. The aggregate blood presence probability score is then used to inform surgeons about the bloodiness of their procedure.

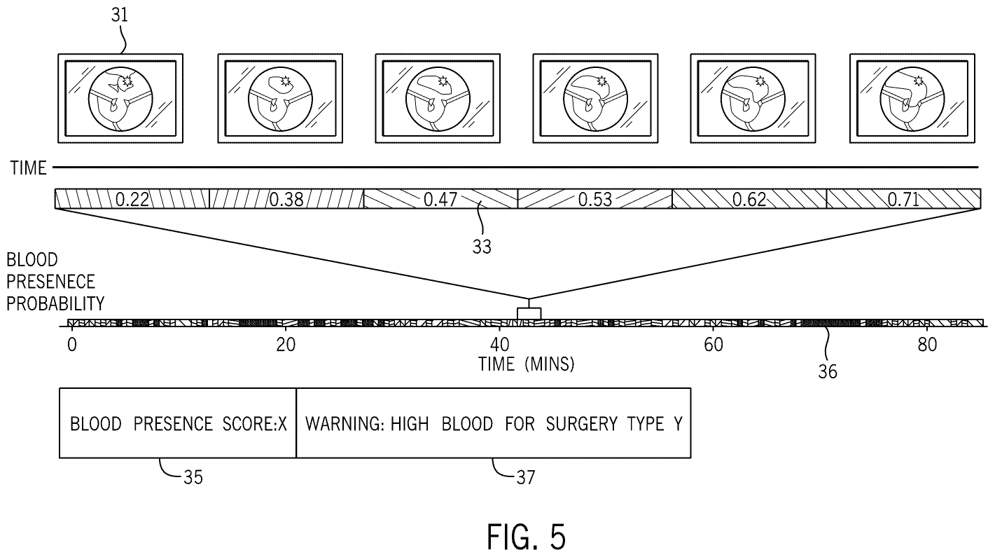

FIG. 5 illustrates a possible display for the output of the model as analyzed by the controller 100. The controller 100 identifies in this example, six frames 31 having associated blood presence values 33. The six frames may be sequential frames in a specific time period identified from a video 36 for the surgical site. The controller 100 calculates a blood presence score 35 according to any of the examples herein. In addition, the controller 100 may generate and display a warning 37 that indicates when the blood presence score 35 exceeds a threshold or expected value. The threshold may be selected according to historical data, a particular user or surgeon, a particular device, or a surgery type.

The controller 100 may also perform the training of the model. The controller 100 may collect ground truth data (e.g., from human annotators) for a plurality of training images. The controller 100 may train the machine-learned model based on the ground truth data.

The controller 100 may identify a surgery type. In one example, an additional neural network or another type of model identifies the surgery type from the video images. The surgery type may be entered into the controller 100 through a user input. The controller 100 associated the images from the surgical site video with the surgery type and assigned the blood presence score with the surgery type.

FIG. 3C illustrates another example of a system for blood presence detection. The system includes at least a camera 101, a learned model 102, hereinafter “model”, and a controller 100. While a memory may be included or connected to the controller 100, an external device 107 also receives data from the controller 100. The external device 107 may be incorporated with and/or provide information to a display 106. In addition, an instrument 105 may send sensor readings or status information to the controller 100. Additional, different, or fewer components may be included.

The external device 107 may be an administrative device or administrative tool running on any computer or mobile device. The external device 107 or the controller 100 may aggregate or log the blood presence score for multiple surgeries or videos to provide assessments of particular surgeons, facilities, teams, instruments, instrument types, or other groupings of surgeries. For example, multiple systems for blood presence detections may be operated at different operating rooms in a facility. Each system may include a controller 100 operable to calculate blood presence scores and relay the information to the external device 107. The external device 107 aggregates the information in order to assess the performance of the facility. The average score or other assessment of the facility may serve as a grade of the facility for ranking among other facilities, government compliance, or self-assessment of average surgical performance.

The external device 107 or the controller 100 may also identify individual medical processionals for the surgery site and provide ranking or other reports of the medical professional based on the blood presence scores. When participating in a surgery, the medical professionals may scan-in to the room via a barcode, RF or other type of reader. Manual logins may be used. Based on this presence information, the controller 100 receive identity information for the medical professionals at the surgery. The controller 100 is configured to associate the images under analysis with the medical professions. The controller 100 further assigns blood presence score with the medical professional.

The controller 100 may evaluate the medical professionals based on the blood presence score. The controller 100 may compare to a user threshold. Each user threshold may be selected based on past scores, manual entry, years of experience, or another factor. Thus, a bloody or not bloody result or a pass or fail type result may be determined based on the threshold.

The external device 107 is configured to aggregate the blood presence scores for multiple medical professionals. The external device 107 may send to display 106 a list of medical professional along with a number of bloody or not blood surgeries. For example, the external device 107 may rank or otherwise assess various surgeons or surgical teams. The external device 107 may generate reports in response to the comparison with the individual thresholds. Each surgical team may be associated with an average or media blood assessment score and provided to display 106. An administrator may provide feedback to individuals based on the feedback. For example, the external device 107 may access instructional information in response to the blood presence score and provide the instructional information to the medical professional through display 106.

The controller 100 may also receive instrument data from instrument 105 and provide results including the blood presence score according to the instrument data. The instrument 105 may transmit identifier information to the controller 100 through wireless communication. The instrument 105 may be scanned in to a surgery using a barcode reader or other reader. The identifier provided to the controller 100 may include a type of the instrument 105 (e.g., stapler, energy device, passive hemostat as described above). The identifier may include a model number, a brand, or a serial number for the instrument 105. The controller 100 (or the learned model 102) may identify the instrument directly from the images. The controller 100 may modify the images collected from the camera 101 according to the instrument data by for example, inserting a header in the image files with the identifier. In addition or in the alternative, the output of the learned model 102 may be modified according to the instrument data by for example, inserting a value in the output data with the identifier. In this way, the controller assigns the blood presence score to the instrument 105.

The controller 100 or the external device 107 may evaluate the instruments based on the blood presence score. The controller 100 may compare to an instrument threshold. The controller 100 may also analyze the output of the model according to the instrument data. The controller 100 or the external device 107 is configured to aggregate the blood presence scores for multiple instruments. The external device 107 may send to display 106 a list of instruments along with a number of bloody or not blood surgeries. For example, the external device 107 may rank or otherwise assess various instrument models or instrument brands. The external device 107 may track an individual instrument's performance overtime to detect degradation. The external device 107 may generate reports in response to the comparison with the instrument thresholds. Each instrument type may be associated with an average or media blood assessment score and provided to display 106. An administrator may select instruments based on the score.

The external device 107 may also correlate the instrument data 105 based on timestamp. The instrument data may not be received directly from the instrument 105 but rather entered into the external device 107 based on inventory or other data entry. The controller 100 or the external device may correlated the usage times of the instrument 105 with the timestamps on the images from the camera 101 or the blood presence scored in other to determine the instrument that is associated with the blood presence scores.

The instrument data may include data values that indicate when the instrument is being used. For example, in the case of the energy device 10, the instrument data may include time values that indicate when the cutting or sealing operations are performed during the surgery. The operations may be indicated by a signal resulting from pressing the handheld trigger of the instrument. The controller 100 may identify the time period for the blood presence score based on the operation performed by the instrument. That is, the time period may correspond to a predetermined length of time after the energy device 10 performs a cutting operation or a sealing operation. In another example, the time period may correspond to a stapling operation of the stapler 11. Thus, the controller 100 may analyze images from the camera to calculate blood presence scores that are associated with individual operations of the instrument.

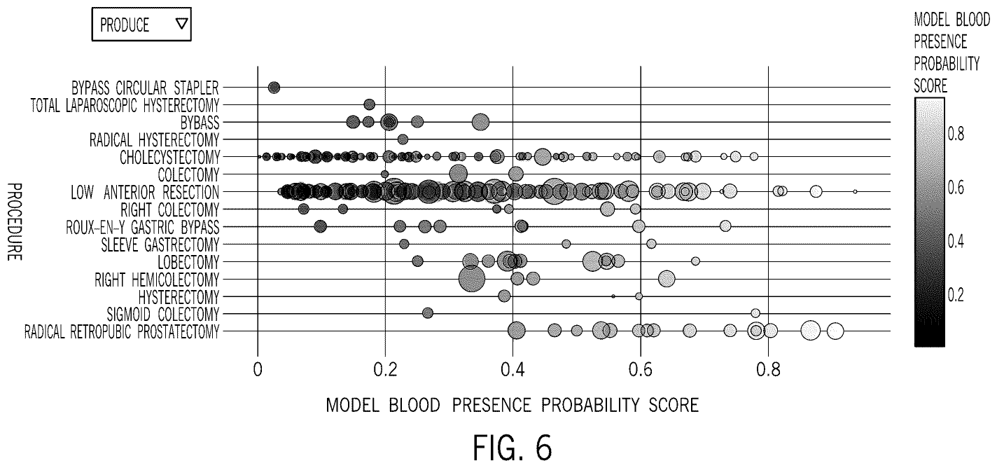

The controller 100 may also identify surgery types based on data entered by the user or assigned to the time slot of the surgery. The controller 100 may derive threshold data (e.g., for comparison of the blood presence score) based on historical data for a particular surgery type. FIG. 6 is a chart of various surgery types and statistical data for blood presence. Example surgery types include bypass circular stapler procedure, total laparoscopic hysterectomy, bypass, radical hysterectomy, cholecystectomy, colectomy, low anterior resection, right colectomy, roux-en-Y gastric bypass, sleeve gastrectomy, lobectomy, right hemicolectomy, hysterectomy, sigmoid colectomy, and radical retropubic prostatectomy. Each surgery type may be associated with a different blood presence threshold. The controller 100 is configured to identify the surgery type and select a threshold based on the identified surgery type. The blood presence score is compared to a threshold for the individual surgery type. Thus, the warning 37 may be issued for a first surgery type with blood presence score X but not issued for a second surgery type with the same blood presence score X.

In another example, an additional or alternative learned model 102 may analyze the images of the surgical site for the location of the bleeding. In this example, the learned model 102 is trained on data for the location of the bleeding in training images as identified by experienced medical professionals. The output of the model may be a region of the patient, which may correspond to image coordinates in the images collected by the camera 101.

The controller 100 receives the output of the model (e.g., image coordinates) and identifies a bleeding locations in the images. The controller 100 may also modify the images to highlight the bleed location. One or more image processing techniques may be performed to identify the bleed location including edge detection, object recognition, invariant feature recognition.

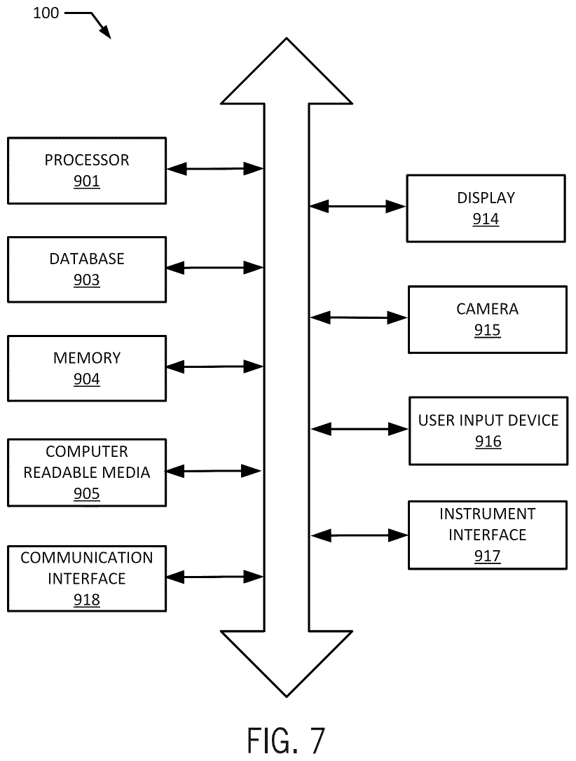

FIG. 7 illustrates an embodiment of a controller 100 the blood presence detection system. The controller 100 may include a bus 910 or other electrical connection facilitating communication between a processor 901 and/or an application specific controller 902, which may be referred to individually or collectively as the controller, and one or more other components including a database 903, a memory 904, a computer readable medium 905, a communication interface 918, a display 914, a camera 915, a user input device 916, and an instrument interface 917. The contents of the database 903 may include any of the data, measurements, or identifiers described herein. The communication interface 918 may be connected to the internet and/or other networks. The other networks may be a content provider server and/or a service provider server. Additional, different, or fewer components may be included.

The communication interface 918 may include any operable connection. An operable connection may be one in which signals, physical communications, and/or logical communications may be sent and/or received. An operable connection may include a physical interface, an electrical interface, and/or a data interface. The communication interface provides for wireless and/or wired communications in any known or later developed format. The communication interface 918 may be connected to the internet and/or other networks. The other networks may be a content provider server or service provider server.

The memory 904 may be a volatile memory or a non-volatile memory. The memory 904 may include one or more of a read only memory (ROM), random access memory (RAM), a flash memory, an electronic erasable program read only memory (EEPROM), or other type of memory. The memory 904 may be removable from the apparatus 900, such as a secure digital (SD) memory card.

The memory 904 and/or the computer readable medium 905 may include a set of instructions that can be executed to cause the controller to perform any one or more of the methods or computer-based functions disclosed herein.

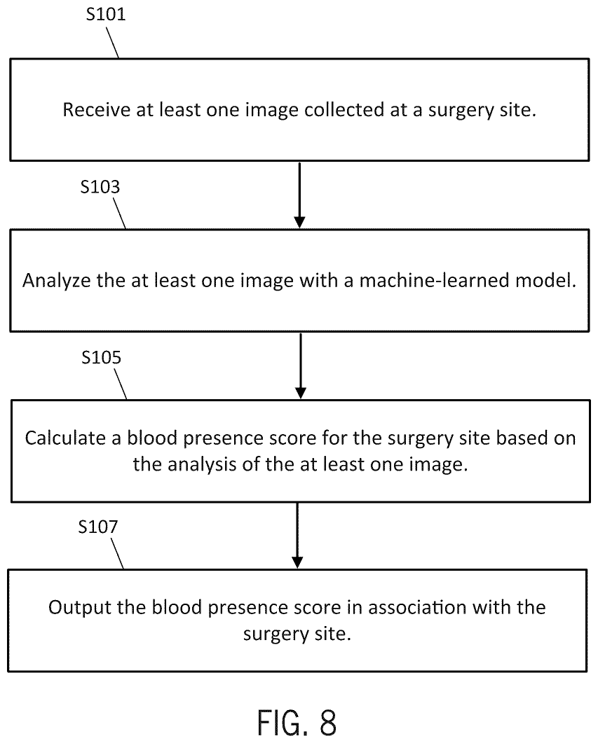

FIG. 8 illustrates an example flow chart for the blood presence detection system of FIG. 7. Any of the embodiments of the system of FIGS. 3A-3C may perform the method. A programmed processor 901 (also referred to here as processing logic or circuit) performs the method. The method may be performed using artificial intelligence. A non-transitory memory (e.g., memory 904) may store instructions for the programmed processor 901 to train and operate the machine-learned model or models. The acts are performed in the order shown or a different order. Additional, different, or fewer acts may be provided.

At act S101, the controller 100 (e.g., processor 901) receives multiple images or a video collected at a surgery site by the camera 915. The images or video may be stored in memory 904. The images or video may be received at the communication interface 918. The images or video are tagged with an identifier for a medical professional. The identifier may be derived from a login that the user enters on a surgical instrument or in user input device 916, a mobile device, or a computer. The identifier may be derived from detection of the medical professional by way of a mobile device using radio frequency identification (RFID), near field communication (NFC), Bluetooth, or another wireless communication. The identifier may be derived through image analysis of a camera of the operating room to identify the medical professional. The identifier may be received at the instrument interface 917.

At act S103, the controller 100 (e.g., processor 901) analyzes the images or video with a machine-learned model such as a neural network. The neural network receives inputs from features of the images or video. The features may include pixel values for brightness, color, intensity or other characteristics of the images or video. In some examples, differences between sequential images are inputs. The neural network is trained on known images that have been labeled as either including blood or substantially absent from blood. Alternatively, the neural network is trained on known images that have been labeled with a varying degree of bloodiness (e.g., 1 to 10). The neural network analyzes subsequent unknown images and outputs a rating that corresponds to the training (e.g., in some implementations including blood or absent from blood and in some instances a degree of bloodiness).

At act S105, the controller 100 (e.g., processor 901) calculates a blood presence score for the surgery site based on the analysis of the images or video. Multiple outputs are combined mathematically to determine the blood presence score. In some examples, the outputs of the neural network for a series of images are averaged to determine the blood presence score. In some instances a count of the number of frames classified as bloody is used as the blood presence score.

At act S107, the controller 100 (e.g., processor 901) outputs the blood presence score in association with the surgery site. The blood presence score may be displayed when it is greater than a threshold value. The threshold value may be based on the average score for a particular type of procedure or for a particular facility. The threshold may be a set number (e.g., 2) of standard deviations above the mean. The threshold may be a set percentage (e.g., 150%) of the mean. The blood presence score may be stored in memory 904 or in the database 903 indexed by time, user, or identifier. The blood presence score may be provided to display 914.

In accordance with various embodiments of the present disclosure, the methods described herein may be implemented by software programs executable by a computer system. Further, in an exemplary, non-limited embodiment, implementations can include distributed processing, component/object distributed processing, and parallel processing. Alternatively, virtual computer system processing can be constructed to implement one or more of the methods or functionality as described herein.

Although the present specification describes components and functions that may be implemented in particular embodiments with reference to particular standards and protocols, the invention is not limited to such standards and protocols. For example, standards for Internet and other packet switched network transmission (e.g., TCP/IP, UDP/IP, HTML, HTTP, HTTPS) represent examples of the state of the art. Such standards are periodically superseded by faster or more efficient equivalents having essentially the same functions. Accordingly, replacement standards and protocols having the same or similar functions as those disclosed herein are considered equivalents thereof.

A computer program (also known as a program, software, software application, script, or code) can be written in any form of programming language, including compiled or interpreted languages, and it can be deployed in any form, including as a standalone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application specific integrated circuit).

As used in this application, the term ‘circuitry’ or ‘circuit’ refers to all of the following: (a) hardware-only circuit implementations (such as implementations in only analog and/or digital circuitry) and (b) to combinations of circuits and software (and/or firmware), such as (as applicable): (i) to a combination of processor(s) or (ii) to portions of processor(s)/software (including digital signal processor(s)), software, and memory(ies) that work together to cause an apparatus, such as a mobile phone or server, to perform various functions) and (c) to circuits, such as a microprocessor(s) or a portion of a microprocessor(s), that require software or firmware for operation, even if the software or firmware is not physically present.

This definition of ‘circuitry’ applies to all uses of this term in this application, including in any claims. As a further example, as used in this application, the term “circuitry” would also cover an implementation of merely a processor (or multiple processors) or portion of a processor and its (or their) accompanying software and/or firmware. The term “circuitry” would also cover, for example and if applicable to the particular claim element, a baseband integrated circuit or applications processor integrated circuit for a mobile phone or a similar integrated circuit in server, a cellular network device, or other network device.

Processors suitable for the execution of a computer program include, by way of example, both general and special purpose microprocessors, and anyone or more processors of any kind of digital computer. Generally, a processor receives instructions and data from a read only memory or a random access memory or both. The essential elements of a computer are a processor for performing instructions and one or more memory devices for storing instructions and data. Generally, a computer also includes, or be operatively coupled to receive data from or transfer data to, or both, one or more mass storage devices for storing data, e.g., magnetic, magneto optical disks, or optical disks. However, a computer need not have such devices. Moreover, a computer can be embedded in another device, e.g., a mobile telephone, a personal digital assistant (PDA), a mobile audio player, a Global Positioning System (GPS) receiver, to name just a few. Computer readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

To provide for interaction with a user, embodiments of the subject matter described in this specification can be implemented on a device having a display, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input.

The term “computer-readable medium” includes a single medium or multiple media, such as a centralized or distributed database, and/or associated caches and servers that store one or more sets of instructions. The term “computer-readable medium” shall also include any medium that is capable of storing, encoding or carrying a set of instructions for execution by a processor or that cause a computer system to perform any one or more of the methods or operations disclosed herein.

In a particular non-limiting, exemplary embodiment, the computer-readable medium can include a solid-state memory such as a memory card or other package that houses one or more non-volatile read-only memories. Further, the computer-readable medium can be a random access memory or other volatile re-writable memory. Additionally, the computer-readable medium can include a magneto-optical or optical medium, such as a disk or tapes or other storage device to capture carrier wave signals such as a signal communicated over a transmission medium. A digital file attachment to an e-mail or other self-contained information archive or set of archives may be considered a distribution medium that is a tangible storage medium. Accordingly, the disclosure is considered to include any one or more of a computer-readable medium or a distribution medium and other equivalents and successor media, in which data or instructions may be stored. These examples may be collectively referred to as a non-transitory computer readable medium.

In an alternative embodiment, dedicated hardware implementations, such as application specific integrated circuits, programmable logic arrays and other hardware devices, can be constructed to implement one or more of the methods described herein. Applications that may include the apparatus and systems of various embodiments can broadly include a variety of electronic and computer systems. One or more embodiments described herein may implement functions using two or more specific interconnected hardware modules or devices with related control and data signals that can be communicated between and through the modules, or as portions of an application-specific integrated circuit.

Embodiments of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), e.g., the Internet.

The computing system can include clients and servers. A client and server are generally remote from each other and typically interact through a communication network. The relationship of client and server arises by virtue of computer programs running on the respective computers and having a client-server relationship to each other

The illustrations of the embodiments described herein are intended to provide a general understanding of the structure of the various embodiments. The illustrations are not intended to serve as a complete description of all of the elements and features of apparatus and systems that utilize the structures or methods described herein. Many other embodiments may be apparent to those of skill in the art upon reviewing the disclosure. Other embodiments may be utilized and derived from the disclosure, such that structural and logical substitutions and changes may be made without departing from the scope of the disclosure. Additionally, the illustrations are merely representational and may not be drawn to scale. Certain proportions within the illustrations may be exaggerated, while other proportions may be minimized. Accordingly, the disclosure and the figures are to be regarded as illustrative rather than restrictive.

While this specification contains many specifics, these should not be construed as limitations on the scope of the invention or of what may be claimed, but rather as descriptions of features specific to particular embodiments of the invention. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings and described herein in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments.

One or more embodiments of the disclosure may be referred to herein, individually and/or collectively, by the term “invention” merely for convenience and without intending to voluntarily limit the scope of this application to any particular invention or inventive concept. Moreover, although specific embodiments have been illustrated and described herein, it should be appreciated that any subsequent arrangement designed to achieve the same or similar purpose may be substituted for the specific embodiments shown. This disclosure is intended to cover any and all subsequent adaptations or variations of various embodiments. Combinations of the above embodiments, and other embodiments not specifically described herein, are apparent to those of skill in the art upon reviewing the description.

The Abstract of the Disclosure is provided to comply with 37 C.F.R. § 1.72(b) and is submitted with the understanding that it will not be used to interpret or limit the scope or meaning of the claims. In addition, in the foregoing Detailed Description, various features may be grouped together or described in a single embodiment for the purpose of streamlining the disclosure. This disclosure is not to be interpreted as reflecting an intention that the claimed embodiments require more features than are expressly recited in each claim. Rather, as the following claims reflect, inventive subject matter may be directed to less than all of the features of any of the disclosed embodiments. Thus, the following claims are incorporated into the Detailed Description, with each claim standing on its own as defining separately claimed subject matter.

It is intended that the foregoing detailed description be regarded as illustrative rather than limiting and that it is understood that the following claims including all equivalents are intended to define the scope of the invention. The claims should not be read as limited to the described order or elements unless stated to that effect. Therefore, all embodiments that come within the scope and spirit of the following claims and equivalents thereto are claimed as the invention.

What is claimed is:

1. A method for bleeding assessment at a surgery site, the method comprising:
receiving at least one image collected at a surgery site;
analyzing the at least one image with a machine-learned model;
calculating a blood presence score for the surgery site based on the analysis of the at least one image; and
outputting the blood presence score in association with the surgery site.

2. The method of claim 1, further comprising:
identifying a time period for the blood presence score, wherein the at least one image includes a plurality of images collected during the time period, wherein the blood presence score is based on an average of output values of the machine-learned model for the plurality of images.

3. The method of claim 1, further comprising:
identifying an instrument for the surgery site;
associating the at least one image with the instrument; and
assigning the blood presence score with the instrument.

4. The method of claim 3, further comprising:
comparing the blood presence score to an instrument threshold; and
reporting instrument performance in response to the comparison.

5. The method of claim 1, further comprising:
identifying a medical professional for the surgery site;
associating the at least one image with the medical professional; and
assigning the blood presence score with the medical professional.

6. The method of claim 5, further comprising:
comparing the blood presence score to a user threshold; and
reporting medical professional performance in response to the comparison.

7. The method of claim 5, further comprising:
accessing instructional information in response to the blood presence score; and
providing the instructional information to the medical professional.

8. The method of claim 1, further comprising:
aggregating blood presence data including the blood presence score for a facility including the surgery site; and
providing an assessment for the facility including a plurality of surgery sites.

9. The method of claim 1, further comprising:
identifying a bleeding location in the at least one image; and
modifying the at least one image to highlight the bleeding location.

10. The method of claim 1, further comprising:
determining one or more timestamps for the at least one image, wherein the one or more timestamps corresponds to a predetermined step of a procedure at the surgical site; and
assigning the one or more timestamps to the blood presence score.

11. The method of claim 10, further comprising:
receiving instrument data associated with one or more instrument timestamps; and
correlating the one or more instrument timestamps with the one or more blood presence timestamps.

12. The method of claim 1, further comprising:
collecting ground truth data for a plurality of training images; and
training the machine-learned model based on the ground truth data.

13. The method of claim 1, further comprising:
identifying a surgery type for the surgery site;
associating the at least one image with the surgery type; and
assigning the blood presence score with the surgery type.

14. The method of claim 1, wherein the at least one image is included in a video of the surgery site.

15. A surgery assessment system comprising:
a camera configured to collect a plurality of images collected at a surgery site;
a processor configured to analyze the plurality of images with a machine-learned model and calculate a blood presence score for the surgery site based on the analysis of the plurality of images; and
a display configured to output the blood presence score in association with the surgery site.

16. The surgery assessment system of claim 15, wherein the display provides a video including the plurality of images in association with the blood presence score.

17. The surgery assessment system of claim 15, wherein output values of the machine-learned model for the plurality of images are averaged to calculate the blood presence score.

18. The surgery assessment system of claim 15, further comprising:
an instrument interface configured to receive instrument data for an instrument associated with the surgery site.

19. The surgery assessment system of claim 15, wherein the display is configured to provide a plurality of blood presence scores for a plurality of surgeries.

20. A surgery assessment system comprising:
a memory configured to store a plurality of images collected at a surgery site; and
a processor configured to analyze the plurality of images with a machine-learned model and calculate a blood presence score for the surgery site based on the analysis of the plurality of images.
